(19) United States
(12) Patent Application Publication
(10) Pub. No.: US 2013/0102013 A1
(43) Pub. Date: Apr. 25, 2013

(54) MATERIALS AND METHODS TO DETECT PYRIMIDINE-PYRIMIDINE DIMER FORMATION

(71) Applicant: Yansong Gu, Bellevue, WA (US)

(72) Inventor: Yansong Gu, Bellevue, WA (US)

(73) Assignee: EMPIRE TECHNOLOGY DEVELOPMENT LLC, Wilmington, DE (US)

(21) Appl. No.: 13/521,894

(22) PCT Filed: Oct. 21, 2011
(86) PCT No.: PCT/US11/57260

§ 371 Date: Jul. 12, 2012

(51) Int. Cl. G01N 33/566; C12M 1/40; G01N 21/64

(52) U.S. Cl. 435/7.92; 436/501; 435/287.2; 422/69

(57) ABSTRACT

Disclosed are methods and apparatuses pertaining to measuring DNA damage due to exposure to ultraviolet light, as measured by formation of pyrimidine-pyrimidine dimers in a polynucleotide. The apparatuses can be in the form of a patch which can be particularly useful in measuring the effectiveness of a sunscreen. The apparatuses can further include a reporter agent for instant determination of the pyrimidine-pyrimidine dimer formation.

### TECHNICAL FIELD

This disclosure relates generally to methods, kits and compositions pertaining to measuring DNA damage due to exposure to ultraviolet light.

### BACKGROUND

The following description is provided to assist the understanding of the reader. None of the information provided or references cited is admitted to be prior art.

DNA is sensitive to radiation exposure, such as ultraviolet (UV) light. UV light is defined as electromagnetic radiation with wavelengths between 100 and 400 nanometers and is a highly energized form of the electromagnetic spectrum. UV light comes to the earth from the sun, though most of the higher energy rays are blocked by the ozone. However, some high energy UV rays still reach the earth and are responsible for sun burns and skin cancer. As the ozone becomes thinner, more of these rays will come through the atmosphere and have a significant effect on human health.

Exposure to UV light can cause formation of pyrimidine-pyrimidine dimers in DNA, which contain covalent links between any adjacent pyrimidines (cytosine, thymine, and uracil) in a DNA strand (FIG. 1). Thymine-thymine dimers are one type of pyrimidine-pyrimidine dimers. Thymine-thymine dimers are relatively stable and have been observed in living systems for up to 3 weeks after UV exposure (Hemminki and Kari (2002) Carcinogenesis 23:605-9). Improper repair of thymine-thymine dimers may result in point mutations in the opposite DNA strand during replication (e.g., G is inserted instead of A). It may even cause more harmful frameshift mutations. Thus, UV radiation can be highly mutagenic and lead to deadly skin cancers.

DNA damage is difficult to measure in living systems as the cellular response and repair to damaged DNA occur rapidly. DNA damage is directly reversible for certain kinds of impairment, such as the enzymatic photoreactivation of thymine-thymine dimers. Photoreactivation is an enzymatic reaction, in which DNA damage induced by exposure to UV radiation is repaired through a sequence of photochemical reactions. Lesions on the DNA strand are recognized by enzymes known as photolyases, followed by the absorption of light wavelengths >300 nm (e.g., fluorescent and sunlight). This absorption enables the photochemical reactions to occur, which results in the elimination of the pyrimidine-pyrimidine dimer, returning it to its original state. However, human cells do not use photolyases to directly reverse UV light-induced lesions. Instead, several mechanisms are in place to detect, remove, and repair this kind of DNA damage, by means of base excision, namely, nucleotide excision repair (NER), base excision repair (BER), and mismatch repair (MMR). These mechanisms splice out the damaged region and insert in its place, new bases to fill the aperture, through DNA polymerization and ligation. Defects in these mechanisms manifest increased sensitivity to UV light and cause several forms of genetic disorders.

To measure UV damage, most techniques on the market measure cell viability and phenotypes after the DNA repair process. These techniques do not quantify the actual DNA lesions directly caused by UV light or sun exposure because the DNA repair mechanism in a cell reduces the amount of DNA damage in the cell and compromises measurement of DNA damage. Varying sensitivities of different individuals to sun exposure also complicate measurement of DNA damage.

There are many devices to measure UV exposure. None of them, however, directly measure DNA damage and there are no simple ways to quantitatively and objectively measure exposure directly to skin. Therefore, there is a need for a method and device for accurate measurement of DNA damage caused by UV exposure.

In addition, various sunscreen products are commercially available. The active ingredients in these products vary, for example, some products use photolyases as the active ingredient, but most of them prevent cell damage by filtering the skin damaging UV rays. In addition, the length of a product sitting on a shelf affects the effectiveness of the product. Currently, there are no accurate ways of measuring the effectiveness of a sunscreen product protection against UV damage on human skin. Therefore, there is a need for a method and device for screening and measuring the sunscreen products for their effectiveness.

### SUMMARY

In one embodiment, the present disclosure provides an apparatus for measuring pyrimidine-pyrimidine dimer formation, the apparatus comprising a scaffold and a polynucleotide disposed on the surface, wherein the polynucleotide comprises a plurality of pyrimidine-pyrimidine dinucleotides.

In some embodiments, the surface is substantially flat. In some embodiments, the pyrimidine-pyrimidine dinucleotides are thymine-thymine dinucleotides. Yet in some embodiments, the pyrimidine-pyrimidine dimer is a thymine-thymine dimer.

In some embodiments, the polynucleotide has an enriched pyrimidine-pyrimidine dinucleotide content as compared to the pyrimidine-pyrimidine dinucleotide content in an average natural polynucleotide. The pyrimidine-pyrimidine dinucleotide content of the polynucleotide, in one aspect, is higher than about 25 pyrimidine-pyrimidine dinucleotides per 100 bases.

The polynucleotide can comprise a single strand polynucleotide or, in an alternative embodiment, a double strand polynucleotide. A non-limiting example of a single strand polynucleotide is a 18-mer poly(T). A non-limiting example of a double strand polynucleotide is a 18-mer poly(T)-poly(A).

In any of the above embodiments, the scaffold can be made of a polymer selected from the group consisting of synthetic rubber, nitrocellulose, cellulose, polyester, aramid, polyurethane, nanosheet, nanoparticle, silk, nylon, PVC, polystyrene, polyethylene, polypropylene, polyacrylonitrile, polysiloxane, polydimethylsiloxane and polytetrafluoroethylene, or combinations thereof.

In another embodiment, the apparatus further comprises an adhesive base, wherein the base is on a side of the scaffold that is opposite to the surface that has the polynucleotide. Still in another embodiment, the scaffold of the apparatus is in the form of a patch. Yet in another embodiment, the polynucleotide is embedded in the scaffold. The embedding, in one aspect, is non-covalent. In another aspect, the embedding allows the polynucleotide to move within or out of the scaffold when in contact with an aqueous solution, such as water, by virtue of capillary effects. In another embodiment, the polynucleotide is conjugated to the scaffold, which can be covalent or non-covalent.

Also provided, in one embodiment of the present disclosure, is a method for measuring pyrimidine-pyrimidine dimer formation, comprising exposing the surface of an apparatus of any of the above embodiments to ultraviolet radiation for a time sufficient to allow for pyrimidine-pyrimidine dimer formation, contacting the surface with an effective amount of an agent that specifically recognizes pyrimidine-pyrimidine dimer under a condition suitable for the agent to bind to the polynucleotide, and measuring the amount of the agent bound to the polynucleotide, thereby determining the pyrimidine-pyrimidine dimer content.

In one embodiment, the agent is an antibody. In another embodiment, the agent is a polypeptide that comprises a pyrimidine-pyrimidine dimer binding sequence. The agent, in some embodiments, comprises a detectable label. In some embodiments, the pyrimidine-pyrimidine dimer is a thymine-thymine dimer.

Another embodiment of the present disclosure provides a method for identifying an inhibitor of pyrimidine-pyrimidine dimer formation, the method comprising exposing the surface of an apparatus of any of the above embodiments to an amount of ultraviolet radiation sufficient to allow for pyrimidine-pyrimidine dimer formation, in the presence or absence of a test inhibitor, contacting the surface with an agent that specifically recognizes pyrimidine-pyrimidine dimer under a condition suitable for the agent to bind to the polynucleotide, and measuring the amount of the agent bound to the polynucleotide, wherein a reduction of the agent bound to the polynucleotide in the presence of the test inhibitor, as compared to in the absence of the test inhibitor, indicates that the test inhibitor is an inhibitor of pyrimidine-pyrimidine dimer formation.

Also provided, in one embodiment, is a method for identifying a blocker of pyrimidine-pyrimidine dimer formation, the method comprising exposing the surface of an apparatus of any of the above embodiments to an amount of ultraviolet radiation sufficient to allow for pyrimidine-pyrimidine dimer formation, in the presence or absence of a test blocker, contacting the surface with an agent that specifically recognizes pyrimidine-pyrimidine dimer under a condition suitable for the agent to bind to the polynucleotide, and measuring the amount of the agent bound to the polynucleotide, wherein a reduction of the agent bound to the polynucleotide in the presence of the test blocker, as compared to in the absence of the test blocker, indicates that the test inhibitor is a blocker of pyrimidine-pyrimidine dimer formation.

In one embodiment, the base of the apparatus is in contact with a human.

In one embodiment, the agent is an antibody. In another embodiment, the agent is a polypeptide that comprises a pyrimidine-pyrimidine dimer binding sequence. In yet another embodiment, the agent comprises a detectable label. In some embodiments, the pyrimidine-pyrimidine dimer is a thymine-thymine dimer.

The present disclosure also provides, in one embodiment, a kit for use in measuring pyrimidine-pyrimidine dimer formation, the kit comprising an apparatus of any of the above embodiments and an agent that specifically recognizes pyrimidine-pyrimidine dimer.

In one embodiment, the agent comprises a detectable label. In another embodiment, the kit further comprises instructions to use the kit to measure pyrimidine-pyrimidine dimer formation.

Methods for measuring pyrimidine-pyrimidine dimer formation are also provided, in some embodiment, comprising exposing a sample comprising a polynucleotide having a plurality of pyrimidine-pyrimidine dinucleotides under conditions suitable for pyrimidine-pyrimidine dimer formation, contacting the sample with an agent that specifically recognizes pyrimidine-pyrimidine dimer under a condition suitable for the agent to bind to the polynucleotide, and measuring the amount of the agent bound to the sample, wherein the amount of the agent bound to the sample is indicative of an amount of pyrimidine-pyrimidine dimer formation.

In one embodiment, the polynucleotide comprises an enriched pyrimidine pyrimidine dinucleotide content as compared to the pyrimidine-pyrimidine dinucleotide content in an average natural polynucleotide. In another embodiment, the pyrimidine-pyrimidine dinucleotide content is higher than about 25 pyrimidine-pyrimidine dinucleotide per 100 bases. In yet another embodiment, the agent comprises a detectable label. Suitable agents that specifically recognize pyrimidine-pyrimidine dimer are described above.

Yet in another embodiment, the method further comprises providing a reference standard to convert an amount of the agent bound to the polynucleotide to an amount of pyrimidine-pyrimidine dimers in the polynucleotide.

The present disclosure provides, in another embodiment, an apparatus for measuring pyrimidine-pyrimidine dimer formation, the apparatus comprising a scaffold having a surface and a polynucleotide, disposed on the surface, having a plurality of pyrimidine-pyrimidine dinucleotides and an agent that specifically recognizes pyrimidine-pyrimidine dimer. In some embodiments, the apparatus comprises a detectable label. In some embodiments, the agent emits a reporter signal upon formation of a pyrimidine-pyrimidine dimer.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 illustrates that ultraviolet light can cause the formation of pyrimidine-pyrimidine dimers in DNA, which contain covalent links between any adjacent pyrimidines (cytosine, thymine, and uracil) in a DNA strand.

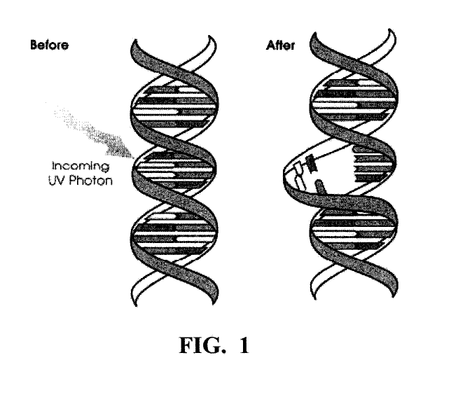

FIG. 2 illustrates the layout of a test strip for detecting pyrimidine-pyrimidine dimers, and method of using it.

FIG. 3 shows the top and size views of the test strip.

FIG. 4 presents an illustrative procedure for manufacturing the test strip.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology is described herein using, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

Nucleotide dimerization is an ordinary occurrence. In every second when a cell is exposed to solar ultraviolet light, approximately 50-100 dimers form in the cell or 50-100 dimers form per 3×10⁹ bases of DNA sequences in mammalian cells in the presence of the DNA damage repair system. The general type of DNA damage is known as a pyrimidine-pyrimidine dimer, which is the adjoining of two identical pyrimidine bases (C, T, or U). An example of a pyrimidine-pyrimidine dimer is a thymine-thymine dimer, which is formed with a cis-syn cyclobutane linkage of the thymine bases. DNA exposed to UV radiation is mainly damaged by the formation of these cyclobutane-type pyrimidine-pyrimidine dimers. Adjacent thymine residues are particularly susceptible to photoreaction, a chemical reaction that involves or requires light.

This disclosure provides methods and apparatuses for determining DNA damage, as measured by formation of pyrimidine-pyrimidine dimers in a polynucleotide, due to exposure to ultraviolet (UV) light. The amount of DNA damage can then be converted to reflect the strength and/or amount of the UV light. Such conversion can be done with a standard curve that correlates formation of pyrimidine-pyrimidine dimers of the polynucleotide with known strength and amount of UV light. In the event the DNA is covered or protected by an UV protection agent such as a sunscreen, the efficacy of the UV protection agent can be determined.

The methods and apparatuses provided in the present disclosure are particularly advantageous in measuring DNA damage due to UV exposure for reasons including, but not limited to, (1) the pyrimidine-pyrimidine dimers formed in the polynucleotides are more stable than those formed in a cell which may be removed or repaired by the DNA repair system in the cell and (2) the polynucleotides are more susceptible to UV exposure compared to a DNA in a cell which is protected by the cell membrane and various cellular structures and molecules. In some embodiments, the methods and apparatuses entail a polynucleotide that contains an enriched pyrimidine-pyrimidine dinucleotide content as compared to the pyrimidine-pyrimidine dinucleotide content in an average natural polynucleotide. Such an enriched pyrimidine-pyrimidine dinucleotide content further increases the sensitivity of the detection as compared to a natural polynucleotide.

### Apparatuses for Measuring Pyrimidine-Pyrimidine Dimer Formation

In one aspect, the present disclosure provides an apparatus for measuring pyrimidine-pyrimidine dimer formation, which apparatus has a scaffold having a surface containing a polynucleotide having a plurality of pyrimidine-pyrimidine dinucleotides. In some embodiments, the surface is substantially flat to allow sufficient exposure to light. In some embodiments, the polynucleotide has an enriched pyrimidine-pyrimidine dinucleotide content as compared to the pyrimidine-pyrimidine dinucleotide content in an average natural polynucleotide. In a particular embodiment, the pyrimidine-pyrimidine dinucleotide is a thymine-thymine dinucleotide. In another embodiment, the pyrimidine-pyrimidine dimer is a thymine-thymine dimer.

### Polynucleotides

As used herein, a “polynucleotide” is understood to be a molecule that has a sequence of bases on a backbone. Bases may be either natural or artificial. Natural bases may include purine and pyrimidine. The purine may be adenine (A) and guanine (G), while the pyrimidine may be thymine (T), cytosine (C), and uracil (U). The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One nucleic acid sequence may be “complementary” to a second nucleic acid sequence. As used herein, the term “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

A polynucleotide can be a natural polynucleotide isolated from a living organism, or a synthesized polynucleotide. In some embodiment, the polynucleotide of the present disclosure may have an enriched pyrimidine-pyrimidine dinucleotide content as compared to the pyrimidine-pyrimidine dinucleotide content in an average natural polynucleotide. The enhanced pyrimidine-pyrimidine dinucleotide content is advantageous for detection of formation of pyrimidine-pyrimidine dimers.

A “pyrimidine-pyrimidine dinucleotide” refers to a pair of pyrimidine bases adjacent to each other in a polynucleotide. The pyrimidine-pyrimidine dinucleotide may be any combination of pyrimidine bases. Representative pyrimidine-pyrimidine dinucleotides include, without limitation, thymine-thymine (T-T) dinucleotides, thymine-cytosine (T-C) dinucleotides, thymine-uracil (T-U) dinucleotides, cytosine-cytosine (C-C) dinucleotides, cytosine-uracil (C-U) dinucleotides, and uracil-uracil (U-U) dinucleotides.

An “enriched pyrimidine-pyrimidine dinucleotide content” as used herein refers to a pyrimidine-pyrimidine dinucleotide content that is higher than the average content of pyrimidine-pyrimidine dinucleotide content in an average natural polynucleotide. The exact average of natural pyrimidine-pyrimidine dinucleotide content is difficult to ascertain but is estimated to be at about 25 pyrimidine-pyrimidine dinucleotide per 100 bases, assuming that all nucleotides evenly occur in a natural nucleic acid. Accordingly, in some embodiments, an enriched pyrimidine-pyrimidine dinucleotide content refers to at least about 30 pyrimidine-pyrimidine dinucleotides, or alternatively at least about 35, or at least about 40, or at least about 45 or at least about 50, or at least about 60, or at least about 70, or at least about 80 dinucleotides, per 100 bases.

An “average natural polynucleotide” as used herein denotes a polynucleotide that includes all types of nucleotides and dinucleotides substantially evenly. Therefore, an average natural polynucleotide includes about 25 thymine per 100 bases, about 25 pyrimidine-pyrimidine dinucleotides per 100 bases, and about 6 thymine-thymine dinucleotides per 100 bases. In this respect, an average natural polynucleotide serves as a control to determine whether a particular polynucleotide of interest has enhanced content of any nucleotide or dinucleotide or combinations thereof.

A pyrimidine-pyrimidine dinucleotide, in some embodiments, is a thymine-thymine dinucleotide. The estimated average of the natural thymine-thymine dinucleotide content is about one thymine-thymine dinucleotide per 16 bases, assuming that all nucleotides evenly occur in a natural nucleic acid. Accordingly, an “enriched thymine-thymine dinucleotide content” refers to at least about one thymine-thymine dinucleotide per 15, or alternatively per 14, per 13, per 12, per 11, per 10, per 9, per 8, per 7, per 6, per 5, per 4, per 3, or per 2 bases. In a particular aspect, the polynucleotide that has an enriched pyrimidine-pyrimidine content includes a 18-mer poly(T) single strand polynucleotide, or a 1.8-mer poly(T)-poly(A) double strand polynucleotide.

In some embodiments, each pyrimidine-pyrimidine dinucleotide in the polynucleotide can have a distance from an adjacent pyrimidine-pyrimidine dinucleotide to allow sufficient space for an agent to recognize or bind each pyrimidine-pyrimidine dimer once the dimer is formed. Accordingly, in one aspect, a pyrimidine-pyrimidine dinucleotide is at least 1, or alternatively at least 2, or 3, or 4, or 5, or 6, or 7, or 8, or 10, or 11, or 12, or 13, or 14, or 15 bases away from an adjacent pyrimidine-pyrimidine dinucleotide.

The amount of polynucleotide in the apparatuses of the present disclosure may vary depending on the characteristics of the polynucleotide such as the pyrimidine-pyrimidine dinucleotide content, the type of agents used to detect the pyrimidine-pyrimidine dimer formation, or the type of the scaffolds. In some embodiments, the amount of polynucleotide in the apparatuses is from about 10⁻¹⁵ μg to about 1 μg per mm². In one aspect, the amount of polynucleotide is from about 10⁻¹² μg to about 0.1 μg per mm², or alternatively from about 10⁻⁹ μg to about 10⁻² μg per mm², or alternatively from about 10⁻⁶ μg to about 10⁻³ μg per mm², or alternatively from 10⁻⁵ μg to about 10⁻⁴ μg per mm².

In another aspect, the polynucleotide can be a single strand polynucleotide or a double strand polynucleotide.

Methods of preparing polynucleotides are known in the art and custom synthesis of pre-designed polynucleotides is commercially available. For example, Geneart, Inc. (Burlingame, Calif.) provides custom polynucleotide synthesis services. Pre-designed polynucleotides, such as poly(T) or poly(T)-poly(A), are also widely available for purchase from vendors such as Life Technologies Corp. (Carlsbad, Calif. 92008 USA). In one embodiment, the polynucleotide is a synthetic polynucleotide. In another embodiment, the polynucleotide is a naturally occurring polynucleotide.

### Scaffolds and Skin Patches

The apparatus can be in the form of a patch, which on the opposite side of the flat surface has an adhesive base suitable for adhering to a surface, such as, but not limited to, clothing, jewelry or skin.

Scaffolds suitable for the apparatuses or the methods of the disclosure can have a substantially flat surface and can provide a solid support for the polynucleotide and are generally known in the art. In one aspect, the scaffold is made of a polymer. Without limitation, examples of the polymer include synthetic rubber, nitrocellulose, cellulose, polyester, aramid, polyurethane, nanosheet, nanoparticle, silk, nylon, PVC, polystyrene, polyethylene, polypropylene, polyacrylonitrile, polysiloxane, polydimethylsiloxane, polytetrafluoroethylene, and combinations thereof.

A “substantially flat surface” as used herein, refers to a surface or a portion of a surface that is not dominated by raised or lowered areas. A substantially flat surface, therefore, allows for exposure of the surface to a light coming from above the surface. In some embodiments, the substantially flat surface includes raised or lowered areas, spikes or holes, so long as the surface is not dominated by them.

In some embodiment, the scaffold has an adhesive base, which in some embodiments, is on the opposite side of the flat surface from the polynucleotide.

In another aspect, the scaffold is in the form a patch. It would be appreciated by one of skill in the art, that the nature of the scaffold and its size and shape can be determined by the end use, for example, an adhesive patch to be worn on clothing, jewelry or skin, or to be carried on a portable device such as a handbag or a stroller. In this regard, the patch can be placed in an area accessible to sunlight, such as on the hat, on the back of a hand or arm, on the front or back of regular clothing or swimsuit, on the outside of a handbag, or on the top of a stroller, and so on. In one aspect, therefore, the patch is non-toxic, soft and breathable, suitable to adhere to skin. In another aspect, the patch is water-resistant. In yet another aspect, the patch has a substantially flat surface that is from about 0.01 to about 10 square inches, or from about 0.1 to about 5 square inches, or from about 0.5 to about 2 square inches, or from about 0.8 to about 1.2 square inches large. The patch can generally have any size and shape suitable for the intended use and environment. Methods of preparing patches are known in the art. For example, United States Patent Application Publication No: 2010/0056972 discloses the composition and methods of making an adhesive patch.

In yet another aspect, the scaffold is in the form of a wearable device. For the purpose of this invention, the wearable device can have a pin or clip or any other mechanism suitable for affixing the device to an object. In one aspect, the wearable device is a ring, a bracelet, a badge, or a pendant.

In some embodiments, the polynucleotide can be embedded, attached, or conjugated to the scaffold, using techniques known in the art. The embedding, attaching or conjugating, in one aspect, is non-covalent, and in another aspect, in covalent. In another aspect, the embedding or attaching allows the polynucleotide, in an aqueous solution, to move within or out of the scaffold by virtue of capillary effects. For example, DNA is commonly bound to nitrocellulose in Southern blots. Similar to nitrocellulose, the polynucleotide can also be attached to natural cellulose, nylon, plastic beads, resins beads, or any of the polymers as disclosed herein.

### Detection of Pyrimidine-Pyrimidine Dimer

Methods of using the apparatus to measure pyrimidine-pyrimidine dimer formation are also provided, the methods including exposing the surface of the apparatus of the present disclosure to ultraviolet radiation for a time sufficient to allow for pyrimidine-pyrimidine dimer formation, contacting the surface with an effective amount of an agent that specifically recognizes pyrimidine-pyrimidine dimer under, a condition suitable for the agent to bind to the polynucleotide, and measuring the amount of the agent bound to the polynucleotide, thereby determining the pyrimidine-pyrimidine dimer content. In another embodiment, detection of the pyrimidine-pyrimidine can be carried out by detecting non-dimerized pyrimidine or pyrimidine-pyrimidine dinucleotides. In this respect, reduction of non-dimerized pyrimidine or pyrimidine-pyrimidine dinucleotides reveals the amount of pyrimidine-pyrimidine dimers formed on the polynucleotide.

In another aspect, the apparatus of the present disclosure can be used to identify an inhibitor or blocker of pyrimidine-pyrimidine dimer formation, the method including exposing the surface of the apparatus of the present disclosure to an amount of ultraviolet radiation sufficient to allow for pyrimidine-pyrimidine dimer formation, in the presence or absence of a test inhibitor or blocker, contacting the surface with an agent directed at pyrimidine-pyrimidine dimer under a condition suitable for the agent to bind to the polynucleotide, and measuring the amount of the agent bound to the polynucleotide, wherein a reduction of the agent bound to the polynucleotide in the presence of the test inhibitor or blocker, as compared to in the absence of the test inhibitor or blocker, indicates that the test agent is an inhibitor or blocker of pyrimidine-pyrimidine dimer formation.

In yet another aspect, the present disclosure provides a method for measuring pyrimidine-pyrimidine dimer formation, including exposing a sample having a polynucleotide that has a plurality of pyrimidine-pyrimidine dinucleotides under conditions suitable for pyrimidine-pyrimidine dimer formation, contacting the sample with an agent that specifically recognizes pyrimidine-pyrimidine dimer under a condition suitable for the agent to bind to the polynucleotide, and measuring the amount of the agent bound to the sample, wherein the amount of the agent bound to the sample is indicative of pyrimidine-pyrimidine dimer formation.

In some embodiments, the pyrimidine-pyrimidine dimer is thymine-thymine dimer. In some embodiments, the agent is an antibody specifically recognizes the pyrimidine-pyrimidine dimer or a polypeptide that contains a pyrimidine-pyrimidine dimer binding sequence.

### Agents and Detection

Pyrimidine-pyrimidine dimers can be recognized by agents that specifically recognize pyrimidine-pyrimidine dimer and the amount of pyrimidine-pyrimidine dimers in a polynucleotide can be determined by measuring the amount of the agent bound to the polynucleotide.

Antibodies that specifically recognize pyrimidine-pyrimidine dimer are commercially available. For example, product number ab28519 from Abeam Inc. (Cambridge, Mass.) is commonly used to detect thymine-thymine dimers. Al-Adhami (2007) Environ Microbiol 73(3):947-55 also discloses detecting thymine-thymine dimers using immunofluorescence microscopy with a monoclonal antibody against thymine-thymine dimers, anti-TDmAb. The amount of antibody bound to the thymine-thymine dimer can then be measured by methods known in the art such as but not limited to ELISA.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. Antibodies can be polyclonal or monoclonal and can be isolated from any suitable biological source, e.g., murine, rat, sheep and canine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

The terms “monoclonal antibody” or “monoclonal antibody composition” as used herein refer to a preparation of antibody molecules of single molecular composition. A monoclonal antibody composition displays a single binding specificity and affinity for a particular epitope.

Polypeptides that contain a pyrimidine-pyrimidine dimer binding sequence can also be used for detection of pyrimidine-pyrimidine dimer formation. For example, McLenigan et al., (1993) Photochem Photobiol. 57(4):655-62 discloses proteins that have pyrimidine-pyrimidine dimer binding sequences which bind to pyrimidine-pyrimidine dimers and their expressions in normal and UV-exposed cells.

In accordance with the methods and apparatuses disclosed herein, the agent may further include a detectable label. Antibodies and polypeptides can be labeled by incorporating moieties detectable by one or more means including, but not limited to, spectroscopic, photochemical, biochemical, immunochemical, or chemical assays. The method of linking or conjugating the label to the antibody or polypeptide depends on the type of label(s) used and the position of the label on the antibody or polypeptide.

As used herein, “labels” are chemical or biochemical moieties useful for labeling a nucleic acid. “Labels” include fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide.

In illustrative embodiments, the antibodies may be labeled with a “fluorescent dye” or a “fluorophore.” As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor® 350; Alexa Fluor® 430; Alexa Fluor® 488; Alexa Fluor® 532; Alexa Fluor® 546™; Alexa Fluor® 568; Alexa Fluor® 594; Alexa Fluor® 633; Alexa Fluor® 647™; Alexa Fluor® 660; Alexa Fluor® 680; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAGT™ CBQCA; ATTO-TAGT™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue®; Cascade Yellow™; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8™; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; IIPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag Fura Red; Mag-Fura-2; Mag-Fura-5; Mag-Indo-1; Magnesium Green; Magnesium Orange; Malachite Green; Marina Blue®; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); Ran; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKI-167; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613[PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin 5; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that are modified to facilitate conjugation to another reactive molecule. As such, fluorescent dyes or fluorophores may include amine-reactive derivatives such as isothiocyanate derivatives and/or succinimidyl ester derivatives of the fluorophore.

Alternatively, the agent that specifically recognizes pyrimidine-pyrimidine dimers can be embedded in the apparatus for easy detection and quick readout. Accordingly, in one embodiment, for any of the apparatus disclosed herein, the apparatus further comprises an agent that specifically recognizes pyrimidine-pyrimidine dimer. In one embodiment, the agent comprises a detectable label. Suitable agents and labels are provided above.

Therefore, when such an apparatus is used, the agent is bound to the pyrimidine-pyrimidine dimer upon formation of the dimer. If the agent has a reporter signal which can be activated upon such binding, then the amount of pyrimidine-pyrimidine dimer can be instantly determined based on the reporter signal emitted from the apparatus. Methods of including reporter signals in diagnosis devices for instant readout are well known in the art. For example, U.S. Pat. No. 7,317,532 discloses various assay result reading devices and methods of preparing and using them.

### Screening Methods

In accordance with some embodiments of the disclosed methods herein, an inhibitor or blocker of pyrimidine-pyrimidine dimer formation can be screened or identified by comparing the pyrimidine-pyrimidine dimer formation in a polynucleotide in the absence or presence of a candidate inhibitor or blocker. Reduced pyrimidine-pyrimidine dimer formation in the presence of the candidate inhibitor or blocker as compared to in the absence of the candidate inhibitor or blocker indicates that the candidate inhibitor or blocker is a suitable inhibitor or blocker of pyrimidine-pyrimidine dimer formation. In one aspect, a reduction of from about 1% to about 100%, from about 2% to about 98%, from about 3% to about 95%, from about 5% to about 90%, from about 5% to about 85%, from about 5% to about 80%, or from about 5% to about 70%, or from about 10% to about 60%, or from about 20% to about 50%, or from about 30% to about 40% of pyrimidine-pyrimidine dimer formation in the presence of the candidate inhibitor or blacker as compared to in the absence of the candidate inhibitor or blocker indicates that the candidate inhibitor or blocker is a suitable inhibitor or blocker of pyrimidine-pyrimidine dimer formation. In another aspect, a suitable inhibitor or blocker reduces pyrimidine-pyrimidine dimer formation by at least about 20%, or about 30%, or about 40%, or about 50%, or about 60%, or about 70%, or about 80%, or about 90%, or about 95%, or about 98%, or at least about 99%. In a particular aspect, a suitable inhibitor or blocker completely blocks pyrimidine-pyrimidine dimer formation. In some embodiments, the candidate agent is a candidate sunscreen, or a sunscreen that is suspected to have expired, or sunscreen serving as positive control.

Various sunscreen products are commercially available. The active ingredients in these products vary. In addition, the length of a product sitting on a shelf affects the effectiveness of the product. Therefore, this method is particularly useful in testing the effectiveness of a sunscreen, or to identify the optimal conditions of using a sunscreen.

### Correlation of Pyrimidine-Pyrimidine Dimer to UV Exposure

DNA damage in a human or other animal subject can be correlated with pyrimidine-pyrimidine dimer formation as measured by the compositions and methods of the present disclosure. Non-human animal subjects can generally be any animal. Example of non-human animals include dogs, cats, cows, horses, pigs, goats, sheep, rats, mice, rabbits, monkeys, moose, squirrel, bear, llama, alpaca, elephant, and so on.

In some embodiments, DNA damage in a subject is measured by pyrimidine-pyrimidine dimers in a tissue sample or urine sample of the subject. A correlation, therefore, can be established by measuring pyrimidine-pyrimidine dimer formation in a composition or method of the present disclosure for a subject and comparing the pyrimidine-pyrimidine dimer formation to the measured DNA damage in the subject. In some embodiment, the correlation is in the form of the correlation equation, or in the form of a standard curve. In some embodiments, the pyrimidine-pyrimidine dimers in a subject are endogenous DNA damage that is not repaired by the endogenous DNA repair system. The presence of unrepaired DNA damage may cause certain cellular response and phenotype.

In some embodiment, formation of pyrimidine-pyrimidine dimers results from UV exposure and the amount of pyrimidine-pyrimidine dimers formed depends on the intensity of the UV light, the wavelength of the UV light, and duration of exposure. A correlation, therefore, can be established by measuring pyrimidine-pyrimidine dimer formation in a composition or method of the present disclosure at different UV exposure. In some embodiment, the correlation is in the form of the correlation equation, or in the form of a standard curve.

Once the correlation between pyrimidine-pyrimidine dimer formation and UV exposure is established, the compositions and methods of the present disclosure then can be used to measure the risk of certain UV exposure, as well as the effectiveness of a 11V protection agent in reducing or mitigating that risk.

### Kits

The materials and components described for use in the methods may be suited for the preparation of a kit. Thus, the disclosure provides kit for use in measuring pyrimidine-pyrimidine dimer formation, including an apparatus of any embodiment of the present disclosure and an agent that specifically recognizes pyrimidine-pyrimidine dimer. In some embodiments, the agent includes a detectable label, or instructions to use the kit to measure pyrimidine-pyrimidine dimer formation. Examples of agents, detectable labels and methods of attaching the detectable label to the agents have been disclosed above.

### EXAMPLES

The present compositions, methods and kits, thus generally described, will be understood more readily by reference to the following examples, which are provided by way of illustration and are not intended to be limiting of the present methods and kits. The following is a description of the materials and experimental procedures used in the Examples.

### Example 1

This example provides a test strip in which the amount of thymine-thymine dimers (TT-dimers) formed due to exposure to ultraviolet (UV) light can be visualized by virtue of color changes due to interaction of the thymine-thymine dimers and a conjugated antibody recognizing them.

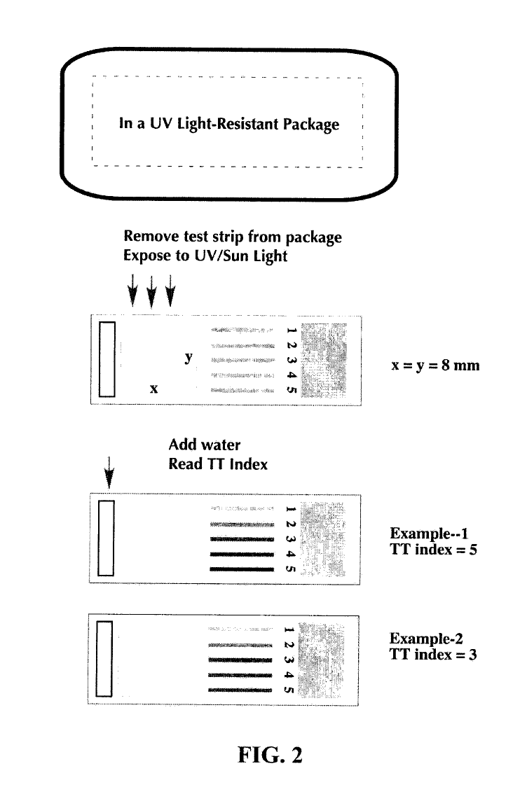

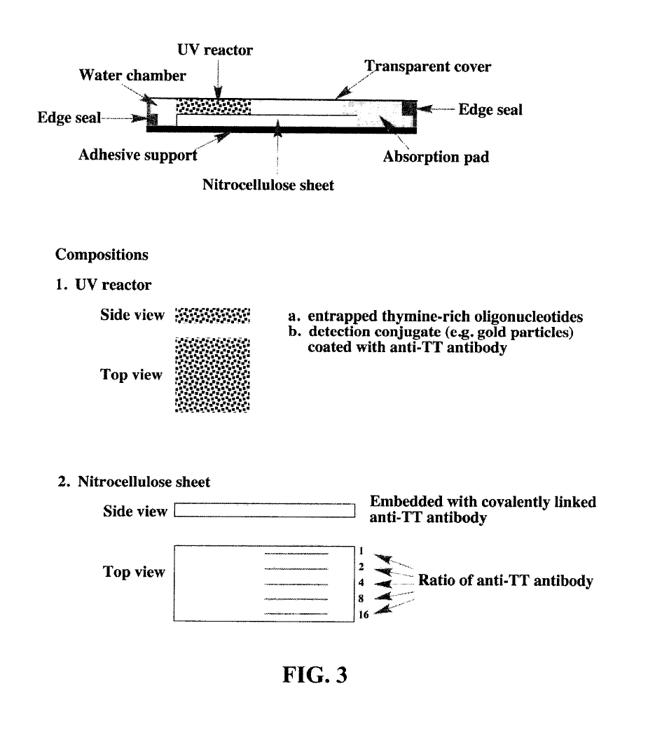

With reference to FIGS. 2 and 3, the test strip includes a water chamber, a UV reactor, a nitrocellulose sheet and an absorption pad. Edge seals are also provided at both ends of the strip.

As shown in FIG. 3, the UV reactor is the scaffold containing oligonucleotides with an enriched thymine-thymine dinucleotide content. The oligonucleotides are entrapped by the porous materials in the UV reactor and can pass through the pores in an aqueous solution. The area of the UV reactor is about 8 millimeter by 8 millimeter.

The nitrocellulose sheet is also made of porous materials. Also as shown in FIG. 3, five thin display lines, each about 6-8 mm in length, are placed in parallel on the surface. Each of the lines is comprised of covalently linked anti-TT-dimer antibodies (e.g., gold particle coated anti-TT-dimer antibodies), but the content of the antibodies vary among the lines. For instance, line 1 has 1 unit of the antibody, line 2 has 2 units, line 3 has 4 units, line 4 has 8 units, and line 5 has 16 units. Thus, each of the lines undergoes color changes when exposed to an aqueous solution, and the final color depends on the concentration of TT-dimers in the solution that interact with the antibodies in the lines.

Varied content of antibodies are embedded in the lines such that each line has a saturation threshold for TT-dimers that migrate across it via microcapillary effects. Accordingly, a TT-index is defined based on such thresholds and can be visualized by the color spectrum generated by the anti-TT-dimer antibody. When the TT dimer concentration increases over a threshold, one or more strips will be saturated and give rise a defined color whereas the other strip(s) may not display saturated color. In this way, the index can be read. For instance, in the event lines 1 to 5 display a full spectrum of red, of which line 1 and 5 correspond to the lowest and highest antibody contents, respectively, such a display of color should correspond to 5 of TT index (see the middle panel in FIG. 2) Likewise, for a TT-dimer concentration that renders lines 1, 2 and 3 pink but lines 4 and 5 red, the TT-dimer concentration can be represented as “TT Index” being 3 (see the bottom panel in FIG. 2). As the TT index reflects the TT-dimer formation in the oligonucleotides due to UV exposure, the TT index is a good indicator of such exposure that may be relevant to other UV indexes.

Also as shown in FIG. 3, the test strip can further include a transparent cover on the top and/or an adhesive support layer on the bottom.

With reference to FIG. 2 again, when the test strip is taken out of its package, it can be worn on a body surface, such as the arm, and thus be exposed to sunlight. After a certain period of time, e.g., about 5, or 15, or 30 minutes, or alternatively about 1 hour, 2 hours or 4 hours the test strip is taken off from the body surface for TT-dimer formation visualization.

For TT-dimer visualization, the water chamber is filled with water and the test strip is placed on a static surface, such as a desk, to allow the microcapillary effect in the absorption pad move the oligonucleotides in the UV reactor through the nitrocellulose sheet where the lines are comprised of the anti-TT-dimer antibodies. Within a few minutes, all of the lines will change colors and the final color will depend on the amount of TT-dimers formed in the oligonucleotides, as illustrated in FIG. 2 and above.

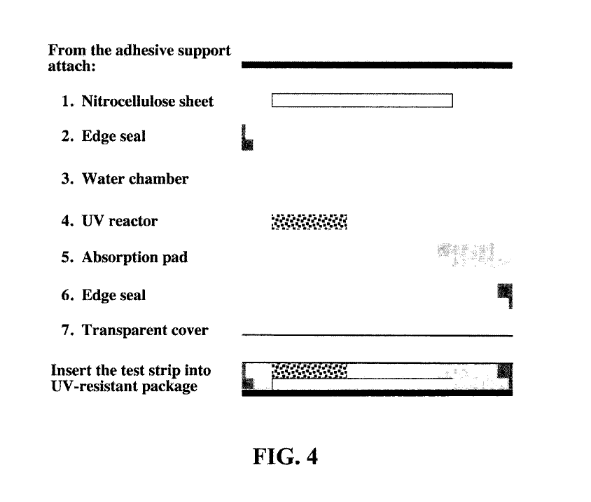

Now referring to FIG. 4, this example also provides a method for manufacturing the test strip. First, an adhesive support is provided. Then, the following components are sequentially placed on the adhesive support: a nitrocellulose sheet, an edge seal, a water chamber, a UV reactor, an absorption pad, another edge seal and finally a transparent cover. Once the test strip is assembled, it can be stored in a UV-proof package.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 particles refers to groups having 1, 2, or 3 particles. Similarly, a group having 1-5 particles refers to groups having 1, 2, 3, 4, or 5 particles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference in their entireties and for all purposes to the same extent as if each individual publication, patent, or patent application was specifically and individually incorporated by reference in its entirety for all purposes.

1. (canceled)

2. The apparatus of claim 8, wherein the surface is substantially flat.

3-7. (canceled)

8. An apparatus for measuring thymine-thymine dimer formation, the apparatus comprising a scaffold having a surface and a polynucleotide disposed on the surface, wherein the polynucleotide is a single strand polynucleotide that comprises a 18-mer poly(T).

9. (canceled)

10. An apparatus for measuring thymine-thymine dimer formation, the apparatus comprising a scaffold having a surface and a polynucleotide disposed on the surface, wherein the polynucleotide is a double strand polynucleotide that comprises a 18-mer poly(T)-poly(A).

11. The apparatus of claim 8, wherein the scaffold is made of a polymer selected from the group consisting of synthetic rubber, nitrocellulose, cellulose, polyester, aramid, polyurethane, nanosheet, nanoparticle, silk, nylon, PVC, polystyrene, polyethylene, polypropylene, polyacrylonitrile, polysiloxane, polydimethylsiloxane, polytetrafluoroethylene, and combinations thereof.

12. The apparatus of claim 8, further comprising an adhesive base, wherein the adhesive base is on a side of the scaffold opposite to the surface.

13. The apparatus of claim 8, wherein the scaffold is in the form of a patch.

14. The apparatus of claim 8, wherein the polynucleotide is non-covalently embedded in the scaffold.

15. A method for measuring thymine-thymine dimer formation, the method comprising:
exposing the surface of the apparatus of claim 8 to ultraviolet radiation for a time sufficient to allow for thymine-thymine dimer formation;
contacting the surface with an effective amount of an agent that specifically recognizes thymine-thymine dimer under a condition suitable for the agent to bind to the polynucleotide; and
measuring the amount of the agent bound to the polynucleotide, thereby determining the thymine-thymine dimer content.

16. The method of claim 15, wherein the agent is an antibody.

17. The method of claim 15, wherein the agent is a polypeptide that comprises a thymine-thymine dimer binding sequence.

18. The method of claim 15, wherein the agent comprises a detectable label.

19. (canceled)

20. A method for identifying an inhibitor of thymine-thymine dimer formation, the method comprising:
exposing the surface of the apparatus of claim 8 to an amount of ultraviolet radiation sufficient to allow for thymine-thymine dimer formation, in the presence or absence of a test inhibitor;
contacting the surface of the apparatus with an agent that specifically recognizes thymine-thymine dimer under a condition suitable for the agent to bind to the polynucleotide; and
measuring the amount of the agent bound to the polynucleotide, wherein a reduction of the agent bound to the polynucleotide in the presence of the test inhibitor, as compared to in the absence of the test inhibitor, indicates that the test inhibitor is an inhibitor of thymine-thymine dimer formation.

21. (canceled)

22. The method of claim 20, wherein the base of the apparatus is in contact with a human.

23. The method of claim 20, wherein the agent is an antibody.

24. The method of claim 20, wherein the agent is a polypeptide that comprises a thymine-thymine dimer binding sequence.

25. The method of claim 20, wherein the agent comprises a detectable label.

26-37. (canceled)
